(19) United States
(12) Patent Application Publication
(10) Pub. No.: US 2025/0342947 A1
(43) Pub. Date: Nov. 6, 2025

(54) PRESERVING DATA INTEGRITY IN TASKS ACROSS A COMPUTING SYSTEM

(71) Applicant: FUJIFILM HEALTHCARE AMERICAS CORPORATION, Lexington, MA (US)

(72) Inventors: Christopher Bartholomew, Bothell, WA (US); Tod Kahler, Kenmore, WA (US); Stuart Nissell, Everett, WA (US); Kathleen Cooper, Everett, WA (US); David Grieco, Everett, WA (US)

(73) Assignee: FUJIFILM HEALTHCARE AMERICAS CORPORATION, Lexington, MA (US)

(21) Appl. No.: 19/265,688

(22) Filed: Jul. 10, 2025

Related U.S. Application Data
(63) Continuation of application No. 17/358,561, filed on Jun. 25, 2021.

(51) Int. Cl. G16H 40/20; G06F 9/48; G06Q 10/1093; G06Q 30/04; G16H 10/60; G16H 30/20

(52) U.S. Cl. CPC G16H 40/20; G06F 9/4881; G06Q 10/1093; G06Q 30/04; G16H 10/60; G16H 30/20

(57) ABSTRACT

Computer-implemented method, computer program products, and computer systems include a processor(s) obtaining a message indicating an individual is present at a location. The processor(s) generates a new service request for the individual with a unique identifier. The processor(s) transmit the new service request to a shared queue in the computing environment.

### PRIORITY CLAIM

This application is a continuation of U.S. patent application Ser. No. 17/358,561, filed Jun. 25, 2021, and the contents which are incorporated by reference herein in its entirety.

### BACKGROUND OF INVENTION

In various service industries, including but not limited to healthcare, movement of a consumer through a workflow, which includes accepting various services from the provider, is managed via an encounter-based workflow that is derived from an order-based workflow. The functionality of systems that manage this workflow relies upon regular manual entries of individual orders, which represent events during an encounter or visit, such as tests or examinations in a medical setting. Plainly, if an order number is not associated with an event, that even cannot be billed. To ensure that orders are created during a given encounter, an employee of the provider must enter and update records throughout the encounter. The manual labor to enter and update these orders is continuous and because it is manual, it is not only inefficient, but it also can potentially introduce errors into the workflow. Common errors include performing the wrong procedure for a given patient, double billing patients for a given procedure, and any human errors that are a result of a physician having to shorten interaction time with a patient because of required time needed for manually entering, updating, and/or correcting data. In one example, in a healthcare setting, a patient exam or procedure is ordered (from an order entry system) before the patient visit. The visit of the patient is assigned an identifier, which can be referred to as an encounter number, and acts as a mechanism by which to identify a patient exam or procedure. However, as the patient progresses through a given visit and various tests and examinations are performed, many of which were arguably not foreseeable in advance of the visit, order numbers must be created for these new tests and examinations. In existing systems, these new orders are assigned temporarily identifiers, but until each temporary identifier is manually updated, for example, by a physician (who places and manually reconciles the orders), the entirety of the encounter cannot be viewed as a whole, unless and until these updates are made.

### SUMMARY OF INVENTION

Shortcomings of the prior art can be overcome and benefits as described later in this disclosure can be achieved through the provision of a method to preserve data integrity of tasks across a computing environment. Various examples of the method are described below, and the method, including and excluding the additional examples enumerated below, in any combination (provided these combinations are not inconsistent), overcome these shortcomings. The method includes: obtaining, by one or more processors in a computing environment, an electronic message indicating an individual is physically present at a service location; generating, by the one or more processors, a new service request for the individual, wherein the new service request for the individual comprises a unique identifier; and transmitting, by the one or more processors, the new service request to a shared queue accessible to one or more modalities comprising resources in the computing environment.

Shortcomings of the prior art can be overcome and benefits as described later in this disclosure can be achieved through the provision of a system to preserve data integrity of tasks across a computing environment. Various examples of the system are described below, and the system, including and excluding the additional examples enumerated below, in any combination (provided these combinations are not inconsistent), overcome these shortcomings. The system includes: a memory; one or more processors in communication with the memory; program instructions executable by the one or more processors via the memory to perform a method, the method including: obtaining, by one or more processors in a computing environment, an electronic message indicating an individual is physically present at a service location; generating, by the one or more processors, a new service request for the individual, wherein the new service request for the individual comprises a unique identifier; and transmitting, by the one or more processors, the new service request to a shared queue accessible to one or more modalities comprising resources in the computing environment.

Shortcomings of the prior art are also overcome and additional advantages are provided through the provision of a computer program product for preserving data integrity of tasks across a computing environment. Various examples of the computer program product system are described below, and the computer program product, including and excluding the additional examples enumerated below, in any combination (provided these combinations are not inconsistent), overcome these shortcomings. The computer program product includes a computer readable storage medium readable by one or more processors and storing instructions for execution by the one or more processors for performing a method comprising: obtaining, by one or more processors in a computing environment, an electronic message indicating an individual is physically present at a service location; generating, by the one or more processors, a new service request for the individual, wherein the new service request for the individual comprises a unique identifier; and transmitting, by the one or more processors, the new service request to a shared queue accessible to one or more modalities comprising resources in the computing environment.

Systems, methods, and computer program products relating to one or more aspects of the technique are also described and may be claimed herein. Further, services relating to one or more aspects of the technique are also described and may be claimed herein.

In some examples, generating the new service request for the individual comprises: determining, by the one or more processors, if an existing service request associated with the individual was generated by the one or more processors during a predefined window of time; based on determining that the existing service request was generated during the predefined window, generating, by the one or more processors, the unique identifier of the new service request based on a unique identifier of the existing service request, the generating comprising: reserving, by the one or more processors, the unique identifier of the existing service request, wherein a final value of the unique identifier of the existing service request comprises an integer; incrementing, by the one or more processors, the final value of the unique identifier of the existing service request by a predefined value; and associating, by the one or more processors, the incremented unique identifier of the existing service request with the new service request as the unique identifier of the new service request.

In some examples, generating the new service request for the individual comprises: determining, by the one or more processors, if an existing service request associated with the individual was generated by the one or more processors during a predefined window of time; and based on determining that no existing service request was generated during the predefined window, generating, by the one or more processors, the unique identifier of the new service request, wherein a final value of the unique identifier of the new service request comprises an integer.

In some examples, the method includes: obtaining, by the one or more processors, a notification from a modality of the one or more modalities that the new service request is complete; reserving, by the one or more processors, the unique identifier of the new service request; and incrementing, by the one or more processors, the final value of the unique identifier of the new service request by the predefined value for use as a unique identifier for a next service request associated with the individual generated during the predefined window of time.

In some examples, the method includes: identifying, by the one or more processors, at least one modality of the one or more modalities, wherein the at least one modality comprises technical characteristics enabling the at least one modality to complete the new service request; and notifying, by the one or more processors, the at least one modality that the new service request was transmitted to the shared queue.

In some examples, transmitting the new service request to the shared queue and notifying the at least one modality are performed in parallel by the one or more processors.

In some examples, generating the new service request for the individual further comprises: collecting, by the one or more processors, metadata from the existing service request; and incorporating, by the one or more processors, the metadata into the new service request.

In some examples, determining if the existing service request associated with the individual was generated by the one or more processors during the predefined window of time comprises querying, by the one or more processors, one or more databases in the computing environment.

In some examples, the window of time is a calendar day when the individual is physically present at the service location.

In some examples, one or more of the electronic message indicating the individual is physically present at the service location and the transmission of the new service request to the shared queue are in a Health Level Seven (HL7) format.

In some examples, the shared queue comprises a modality worklist.

In some examples, the predefined value is 1.

In some examples, the integer of the new service request comprises an accession number, wherein the accession number of the new service is a number in an ordered sequence of services requested for the individual during the predefined window.

In some examples, the unique identifier of the new service request comprises a concatenation of values, the values selected from the group consisting of: the service location, a visit number for the individual at the service location, an identifier of the individual, and an identifier of the service.

In some examples, the electronic message indicating the individual is physically present at the service location does not comprise data or metadata identifying the individual, the method further comprising: associating, by the one or more processors, the new service request with a medical record number in an electronic medical records system in the computing environment; and updating, by the one or more processors, the new service request based on the associating.

In some examples, the method further comprises: messaging, by the one or more processors, an electronic billing system, to instruct the electronic billing systems to generate a bill for the completed new service request.

Additional features are realized through the techniques described herein. Other examples and aspects are described in detail herein and are considered a part of the claimed aspects. These and other objects, features and advantages of this disclosure will become apparent from the following detailed description of the various aspects of the disclosure taken in conjunction with the accompanying drawings.

It should be appreciated that all combinations of the foregoing aspects and additional concepts discussed in greater detail below (provided such concepts are not mutually inconsistent) are contemplated as being part of the inventive subject matter and to achieve the advantages disclosed herein.

### BRIEF DESCRIPTION OF DRAWINGS

One or more aspects of the present invention are particularly pointed out and distinctly claimed as examples in the claims at the conclusion of the specification. The foregoing and objects, features, and advantages of one or more aspects of the invention are apparent from the following detailed description taken in conjunction with the accompanying drawing.

FIG. 1 depicts a workflow that can be utilized by an existing system.

FIG. 2 depicts various aspects of a workflow of some embodiments of the present invention.

FIG. 3 depicts various aspects of a workflow of some embodiments of the present invention and certain elements of a technical architecture of a computing environment.

FIG. 4 depicts various aspects of a workflow of some embodiments of the present invention.

FIG. 5 depicts various aspects of a workflow of some embodiments of the present invention.

FIG. 6 depicts various aspects of a technical environment into which aspects of some embodiments of the present invention can be implemented.

FIG. 7 depicts a computer system configured to perform an aspect of an embodiment of the present invention.

FIG. 8 depicts a computer program product incorporating one or more aspects of the present invention.

### DETAILED DESCRIPTION OF THE INVENTION

Aspects of the present invention and certain features, advantages, and details thereof, are explained more fully below with reference to the non-limiting examples illustrated in the accompanying drawings. Descriptions of well-known materials, fabrication tools, processing techniques, etc., are omitted so as not to unnecessarily obscure the invention in detail. It should be understood, however, that the detailed description and the specific examples, while indicating aspects of the invention, are given by way of illustration only, and not by way of limitation. Various substitutions, modifications, additions, and/or arrangements, within the spirit and/or scope of the underlying inventive concepts will be apparent to those skilled in the art from this disclosure. The terms software and program code are used interchangeably throughout this application and can refer to logic executed by both hardware and software. Components of the system that can be utilized to execute aspects of embodiments of the present invention may include specialized hardware, including but not limited to, an FPGA and a GPU (graphics processor unit). Additionally or alternatively, items denoted as processors may include hardware and/or software processors or other processing means, including but not limited to a software defined radio and/or custom hardware.

As noted above, as understood by one of skill in the art, program code, as referred to throughout this application, includes both software and hardware. For example, program code in certain embodiments of the present invention includes fixed function hardware, while other embodiments utilize a software-based implementation of the functionality described. Certain embodiments combine both types of program code. One example of program code includes a program/utility, having a set (at least one) of program modules, stored in a memory. The terms “including” and “comprising”, as used herein, mean the same thing.

The terms “substantially”, “approximately”, “about”, “relatively”, or other such similar terms that may be used throughout this disclosure, including the claims, are used to describe and account for small fluctuations, such as due to variations in processing, from a reference or parameter. Such small fluctuations include a zero fluctuation from the reference or parameter as well. For example, they can refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. If used herein, the terms “substantially”, “approximately”, “about”, “relatively,” or other such similar terms may also refer to no fluctuations, that is, ±0%.

The terms modality, system, application, and computing node are used herein to represent various resources of a computing system. In the examples herein, a queue or worklist can be shared by these resources and tasks in the queue or worklist can be completed by one or more of the various resources. For the purposes of this application, the terms “modality” and “system” are used interchangeably.

Embodiments of the present invention include computer systems, computer program products, and computer-implemented methods in which program code executed by one or more processors automatically generates, stores, and updates various elements of electronic records in a point-of-care or point-of-service computer system, in a secure and efficient manner. As will be described in greater detail herein, in embodiments of the present invention, program code executing on one or more processors: 1) automatically creates and populates accession numbers that are related to a given encounter (e.g., visit); 2) dynamically increments orders numbers and accession numbers to generate unique identifiers for each to achieve uniqueness; and 3) interfaces with legacy devices and security utilized in a given point-of-care or point-of-service computer system (e.g., a point of care ultrasound (POCUS) system) such that worklist programs executed on these devices can automatically retrieve consumer (e.g., patient) records with task assignments (e.g., studies, tests, procedures, etc.) that include unique identifiers.

Embodiments of the present invention are inextricably tied to computing because aspects of the examples herein improve the performance of a complex computing system. Aspects of embodiments of the present invention are integrated into environments where various systems, including legacy systems, are synchronized to function effectively and efficiently. Thus, embodiments of the present invention utilize a messaging system and a shared queue to liaise between these systems, which accomplish different technical tasks. In generating these tasks, the program code ensures that each task has a unique identifier and represents an actual task to be completed by a modality in the computing environment. Preserving data integrity is a motivator in implementing aspects of the present invention in place of existing systems, which include manual entry and manual reconciliation of data. Common data-related errors that occur in existing systems include, but are not limited to, performing the wrong procedure for a given patient, double billing patients for a given procedure, and/or any human errors that are a result of a physician having to shorten interaction time with a patient because of required time needed for manually entering, updating, and/or correcting data. Additionally or alternatively, existing systems generate records for services or tasks that are not completed and there is no mechanism for cleaning resources of the computing system of excess unused records, which can compromise system performance. Aspects of the examples herein do not produce excess unused records.

Aspects of some embodiments of the present invention represent practical applications. Generally, data accuracy and efficient resource performance is of particular importance in service environments, including the healthcare environments into which aspects of some embodiments of the present invention are implemented. Specifically, implementing aspects of some embodiments of the present invention improves data integrity and consistency within the computing environments into which these aspects are implemented. These computing environments can include disparate systems (including legacy systems) and effectively managing tasks completed by the various systems for a common order, operating within the security policies if the computing environments, and preserving data consistency and integrity of the tasks, are practical applications performed by the aspects described herein.

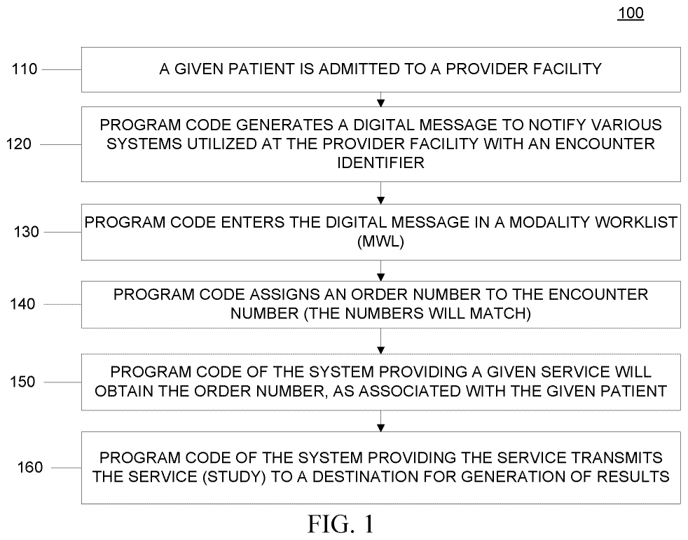

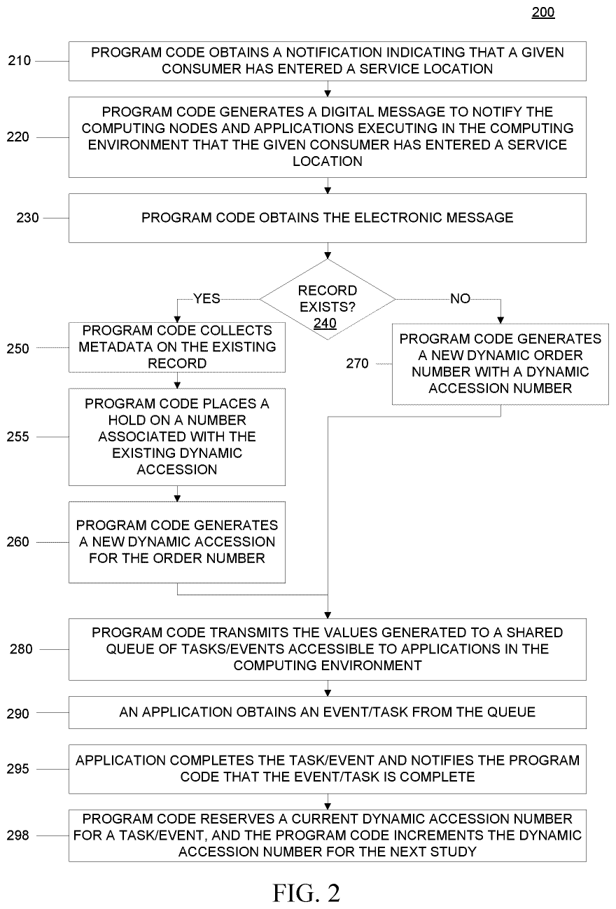

Aspects of various embodiments of the present invention represent significant improvements over existing methods of generating, storing, and updating various elements of electronic records in a point-of-care or point-of-service computer system. Existing methods for workflow management of events during a given service opportunity create resource inefficiencies downstream. In these existing systems, a large demand for resources is created downstream. For example, as will be described with reference to FIG. 1, events are manually updated continuously to enable completion of the workflow (with billing the events). Additionally, unused orders remain in the system unless specifically cleaned out, potentially straining resources. These inefficiencies introduce various risks into systems that secure personally identifiable information. Thus, eliminating these inefficiencies by limiting unnecessary interactions, such as manual data manipulation and cleanup, which are both central to the continued functionality of existing systems, improve the security of the present system. Differences between present methods and aspects of the embodiments disclosed herein are further illustrated in FIGS. 1-4. FIG. 1 is a workflow 100 of an existing event management system for a point-of-care or point-of-service computer system or application. FIGS. 2-4 illustrates various aspects of workflows 200, 300, 400 that illustrates various aspects of some embodiments of the present invention.

FIG. 1 depicts a workflow 100 that can be utilized in an existing encounter management system. Workflows such as that illustrated in FIG. 1 necessitate administrative work downstream to compensate for inefficiencies upstream. As discussed above, program code in embodiments of the present invention, as well as in existing systems, can interface with a billing system as part of generating records for events during a given encounter. This interface is enabled by a unique order number representing each event (e.g., service, task, study, test, consultation, procedure, examination, etc.). In the workflow 100, which can be characterized as an encounter-based workflow, an encounter acts as a temporary order number. The encounter is the identifier until an individual, such as a physician in the case of a point of care system, updates the record and generates an order (with details of an event experienced by the consumer during the visit or encounter). In existing systems, for each event experienced by a consumer (e.g., test or examination performed on a given patient), in certain medical point of care systems, a physician will spend on average five (5) minutes not only placing orders, but also manually reconciling a service provided such as a study with the newly created order numbers. Certain inefficiencies within these systems render them particularly inefficient and require manual intervention, for example, multi-study scans that are done on a single encounter cannot be billed separately. Thus, a physician or other individual must manually split the study to enable the interface with the billing system to function effectively. As aforementioned, in the medical context, existing methods include a significant amount of manual entry, which translates not only to lost time and financial resources, but also, into errors that can affect patient care. A common error in existing systems is that patients are double billed as often, a given scan is entered twice as both an educational and a procedural study. Patients are not billed for educational studies, but unless a manual adjustment is made to one of the entries, both will be billed, double billing the patient and creating a record duplication. Although billing errors are annoying, of arguably greater importance is that data errors in existing systems can result in the wrong procedure being performed on a given patient. Because manual labor in required downstream to enter and update studies (e.g., events, exams, scans, etc.), a physician may manually enter a group of studies for different patients. Because these orders are reconciled to a patient visit later in a visit, the wrong study can be performed on an individual and this error is only discovered when a medical professional interprets the study results. In one example, a physician enters a group of studies, including a cardiac and lung studies and the cardiac study is performed on a patient who needed a lung study because the patient is scanned against the wrong study. A medical professional responsible for interpreting the results see the wrong scan and does not know whether the scan he/she/they is being asked to interpret is for the wrong patient or if the patient data is correct, but the wrong study was performed. The administrative burden on the physician downstream is large and can impact the time the physician spends with the patient, but as seen by this example, entering multiple studies in advance to attempt to lessen the administrative load downstream can lead to data inconsistencies that compromise quality patient care.

FIG. 1 provides the workflow 100 that can be utilized by an existing system within the context of a medical point-of-care system. As illustrated in FIG. 1, an issue with this existing system, which is addressed by embodiments of the present invention, is that a single order number will be created for a given event (e.g., procedure). Thus, when a given event comprises multiple sub-events, which is often the case, there is no automated, efficient, and secure functionality within the system to reflect this reality in a manner that can be transmitted to a billing system or accurately reflected in the point-of-care system itself. (The creation of many sub-events from an initial event is particularly prevalent in an emergency medicine environment where a given individual will present at an emergency room and may be navigated through many departments (which perform different procedures) during a given visit.) As noted above, manual labor is required downstream to correct this deficiency. As illustrated in FIG. 1, the workflow 100 commences when a given patient is admitted to a provider facility, such as a hospital (110). Based on the admission record, program code generates a digital message (e.g., in a Health Level Seven or HL7 format in accordance with international standards for transfer of clinical and administrative data between software applications) to notify various systems utilized at the provider facility with an encounter identifier (120). Program code enters the digital message in a modality worklist (MWL) (130). As understood by one in a medical field, an MWL is a service of digital imaging and communications in medicine (DICOM) systems that makes patient demographic information from various medical systems available at a modality, eliminating dual data entry and providing data integrity. For example, the MWL is an apparatus that provides ultrasound patient metadata through DICOM standards. Thus, a modality, such as an ultrasound system, can obtain patient metadata via the MWL. The program code of the existing systems assigns an order number to the encounter number (the numbers will match) (140). Thus, when communicating via the MWL, the program code of the system providing a given service will obtain the order number, as associated with the given patient (150). The program code of the system providing the service (e.g., an ultrasound system) transmits the service (study) to a destination (e.g., system) which can generate results (160). Using the example of the ultrasound systems, the program code of the system providing the service transmits the service (study) to a picture archiving and communication system (PACS) archive for generation of results. As noted above, once the results for a given order are created, the service associated with the order can be billed (via an interface with a medical billing system).

In FIG. 1, the order number generated by the program code (140), is associated, by the program code, with an original service (e.g., study) that was completed (160). As understood by any individual who visits a service provider (e.g., hospital, healthcare facility, mechanic, etc.), the initial service or reason for a visit may not match the services received at the end of the visit or encounter. Using medical services as an example, various tests and scans can be requested by a professional based on various occurrences during an interaction, which were unanticipated until aspects of these interactions necessitated these tests and scans, in the professional opinion of the service provider. As aforementioned, interfacing with a billing system does not work unless each service has its own order number. Thus, when unanticipated services are rendered, a user must manually adjust events in the point-of-care system (e.g., additional ordering, splitting, and merging studies). In addition to the data integrity and processing efficiency issues discussed above, this manual intervention also introduces challenges and uncertainties including creating a risk that by adding additional services or studies, the original studies that were performed, will not be billed.

An example of the lack of flexibility and need for manual intervention in existing systems is illustrated in radiology orders which commence with a visit to an emergency department. This example is provided for illustrative purposes only and not to suggest any limitations to the benefits of aspects of the present invention over existing systems. In existing orders-based radiology systems, a patient is scheduled for an exam in advance of a visit. The exam is created in the system and is updated, e.g., by a physician within an emergency department, before the data from the visit can be transmitted to a billing system. The exam that is created is specific to an order; it is a single result. Thus, if there are further examinations or procedures completed during the given visit, each event and/or study will attach to the original order by the program code. The limitation of this association causes large overhead for the customer as any study that is not applicable to the original order will need to be curated for it to be correctly billed. Each additional study must be split off through manual intervention, which may include: placing orders for individual additional studies, reconciling each study after it is completed, and/or generating result documentation for each study to finalize billing. Managing these orders manually presents data integrity challenges including, but not limited to: 1) managing the MWL can be impacted when patients are no longer registered in the department where a visit commenced (e.g., the emergency department) or have acquired new encounter numbers from a later visit after being discharged; 2) managing duplicate entries when patients are still present on the worklist, but a user making a manual entry selected an incorrect encounter, and the new study is applied to a patient who was earlier discharged that day.

In embodiments of the present invention, as illustrated in the workflow 200 of FIG. 2, the issues created by the correspondence between the initially generated order number and the event does not create a limitation that requires user intervention to address. Rather, in embodiments of the present invention, a given order is associated, automatically, by the program code, with one or more accession numbers. In this context, an accession number is a child order, also understood as a filled order. In existing encounter-based systems, an order and an accession number are recognized, by the program code, as being the same. As interconnectivity between various systems within a medical environment increase, the flexibility provided by the automatic creation of unique accession numbers, which are associated, by the program code, with order numbers, enables the collaboration of various systems which handle different possible events which could comprise a singular visit for an entity. Specifically, embodiments of the present invention are compatible with HL7 messaging and utilization of an MWL.

Referring to FIG. 2, in an embodiments of the present invention program code executing on one or more processors in a computing environment obtains a notification indicating that a given consumer has entered a service location (210). In this context, this entry in the service location or encounter will be used, as further illustrated in FIG. 2, by the program code to generate orders and accession numbers that are related to this visit. Returning to the notification (210), for example, in a hospital setting, the program code obtains a notification indicating that a given patient has been admitted to the hospital. Based on obtaining this notification (trigger), the program code generates a digital message (e.g., through HL7 in a medical setting) to notify the computing nodes and applications executing in the computing environment that the given consumer has entered a service location (220). In some embodiments of the present invention, the program code generates the digital message but there is no notification sent to the computing nodes and applications. Rather, the program code of an embodiment of the present invention obtains the electronic message (230), but the computing nodes and applications are not alerted to the existence of the digital message until the program code has associated the task or service request in the digital message with a dynamic order and accession item. In a medical environment, utilizing existing HL7 messaging standards comports with security protocols within the computing environment as the notification can include personally identifiable information. Program code in an embodiments of the present invention obtains the electronic message (230).

In some embodiments of the present invention, the notification and message are obtained (210) and generated (220) by applications outside of embodiments of the present invention and program code in embodiments of the present invention interacts, via the digital messaging, with these outside applications in the computing environment. However, in some embodiments of the present invention, obtaining the notification and/or generating the digital message can be executed by program code in embodiments of the present invention. Aspects of some embodiments of the present invention are integrated into existing computing environments and interact with legacy systems, thus providing dynamic functionality within security and technological constraints of the existing computing environments. Therefore, depending on the existing technical architecture, obtaining the notification (210) and/or generating the digital message (220) may or may not be a function of the program code disclosed in the examples herein.

Program code in embodiments of the present invention generates and assigns unique identifiers to each accession (sub-record) for a given encounter (order). The program code increments orders and accession numbers to achieve this uniqueness. Thus, even if multiple services are provided as part of a given event, the program code can effectively and automatically split the event (e.g., study), unlike in existing systems. (As will be discussed later herein and as illustrated in FIG. 2, the program code can reserve a next available dynamic accession number and automatically assigns it to a new even/service/study without user intervention.).

In some embodiments of the present invention, the dynamic order numbers generated by the program code include a unique number of a predefined maximum number of characters. This value includes a prefix that can include a concatenation of various values or descriptive parameters, including but not limited to, the location visited and an identifier for the visit itself. For example, a dynamic order number may comprise a maximum capacity of 22 characters in length that contains any displayable (printable) ACSII characters (hexadecimal values between 20 and 7E, inclusive, or ASCII decimal values between 32 and 126), and comprising of a concatenation of a “prefix” for the department with the visit/encounter identifier (e.g., US112,233 with “US” standing for “ultrasound” and 112233 denoting the encounter identifier). As discussed above, accession numbers are children of a given order number. In the context of the examples provided herein, a dynamic accession number is also a unique number of a predefined maximum length. For example, a dynamic accession number, like the dynamic order number, may be a unique number with a maximum capacity of 22 characters in length that contains any displayable (printable) ACSII characters (hexadecimal values between 20 and 7E, inclusive, or ASCII decimal values between 32 and 126), and comprising of a concatenation of a “prefix” for the department, the visit/encounter identifier, a symbolic type character such as a dash (-) or period (.), and the current study count, starting with the number 1 (e.g., US112233.1 or US112,233-1, denoted as [-/.]).

Various nomenclatures can be utilized by the program code in embodiments of the present invention to generate dynamic order numbers with associated dynamic accession numbers. For example, an order with an accession number can also take the following format: <INDIVIDUAL IDENTIFIER PREFIX><ENCOUNTER IDENTIFIER> [-/.] <ACCESSION NUMBER>. In a medical setting, the individual identifier can be a patient prefix. The characters -/. are utilized herein as non-limiting examples of character dividers that may or may not be implemented to dynamic accession numbers generated by the program code such that each dynamic accession number is unique and a child of an order number.

Returning to FIG. 2, based on obtaining the electronic message (230), program code executing on one or more processors, determines if there is a record for the given consumer that was generated within a defined window of time (240). For example, in embodiments of the present invention that are integrated into medical systems at hospitals, the program code may or may not check to see if given individual, who is a patient, has an encounter on the same day as the program code obtained the electronic message. As discussed above, in existing systems, manual users can sometimes accidentally create duplicate records, which compromises data integrity. Thus, in embodiments of the present invention, the program code automatically checks for any duplication and thus prevents this issue. In some embodiments of the present invention, the program code checks for existing records in one or more databases accessible to the program code in the computing environment.

If the program code determines that a record for the given consumer within the defined window of time already exists, the program code collects metadata on the existing record (which is already associated with a dynamic accession identifier) (250). Additionally, if the program code determines that the record for the given consumer within the defined window of time already exists, the program code places a hold on the number (identifier) associated with the existing dynamic accession (255). By placing this hold, the program code closes out the dynamic accession. For example, if a currently available number (unique identifier for an order) is US112233[-/.]2, the program code reserves “[-/.]2” by removing it from the pool of unique identifiers it can assign. In some embodiments of the present invention, each unique order number with a concatenated accession number will start with at least a “[-/.]1” value. As noted in this example, the program code closes out a given accession number for an order at its completion. However, in various service provider environments, various tasks may be accomplished by different systems in parallel. Thus, a given record could exist that has not been resolved by the system. Thus, the program code reserves (255) its accession number upon locating the record so that it cannot be duplicated and its completion and ultimate billing through the computing environment can occur smoothly.

Returning to this example, also, if the program code determines that the record for the given consumer within the defined window of time already exists, the program code generates a new dynamic accession number and attaches that dynamic accession number to the existing dynamic order number (e.g., US112233[-/.]3) (260). Thus, an order number, US112233, has become dynamic and is no longer tied to a single event during a given encounter, based on the program code augmenting the order with individual identifiers. By placing the hold (255), the program code eliminates any duplication among accession numbers (which are children of a given order number), and by incrementing the number utilizing an identifier (260), the program code creates a unique accession number for a given event during the encounter.

Alternatively, if the program code determines that a record for the given consumer within the defined window of time does not exist, the program code generates a new dynamic order number with a dynamic accession number (270). For example, the program code can generate the dynamic order number of US112233, and this order number, with the dynamic accession number, would be US112233[-/.]1, as generated by the program code.

As illustrated in FIG. 2, the program code transmits the values generated to a shared queue of tasks/events accessible to applications in the computing environment (280). As illustrated in FIG. 2, the contents (and nomenclature) of the items in the queue depend on whether there was an existing consumer record. In one of the examples (e.g., FIG. 2, 250, 255, 260), the program code transmits US112233[-/.]3 to the queue while in another of the examples, the program code transmits US112233[-/.]1 (e.g., FIG. 2, 270) to the queue. In a healthcare setting, the shared queue can be an MWL and the program code of an embodiment of the present invention serves the generated list of dynamically generated accession numbers to the MWL. In this healthcare setting, the MWL is accessed by applications (e.g., the modalities within the environment) and/or messages applications in the computing environment utilizing DICOM standards with metadata of the given consumer/patient. The utilization of a shared queue (e.g., MWL) by the program code enables dynamic orders associated with various consumers to travel with them as they navigate a location and receive various services. The dynamic accession numbers are not duplicated by the program code and despite the services received or the order of those services, the ordering and uniqueness of the dynamic accession numbers is maintained.

Once a task/event is added to the shared queue, applications and devices in the computing environment which can complete these tasks can obtain the tasks (relevant data and the requests) in varied manners, across various embodiments of the present invention. In certain embodiments of the present invention, various applications within the computing environment poll the queue at given intervals to receive lists of accessions for completion (e.g., a given modality could receive requests to complete events/tasks for different individuals). In some computing environments, various applications interface with the queue and an interface that displays the relevant worklist items updates in real-time, alerting the modalities to the relevant worklist or queue items. Additionally or alternatively, program code in an embodiments of the present invention notifies the relevant applications or modalities which can handle various tasks or the creation of the tasks in the queue. Messages generated, transmitted, and/or received during these interactions can utilize transport layer security (TLS), in some embodiments of the present invention.

The worklist or queue that the applications and computing nodes in the computer environment (e.g., Point of Care Ultrasound (POCUS) devices) access to retrieve consumer events/tasks provides a next event (e.g., study) for the consumer (e.g., patient). As illustrated in FIG. 2, an application obtains an event/task from the queue (290). Returning to the medical setting example, the MWL service can send a digital message to an application in the computing environment and/or the application can check the MWL for messages. In both examples, the digital message is obtained, for example, by program code of an ultrasound system, with a current dynamic accession number for the patient. The application completes the task/event and notifies the program code that the event/task is complete (295). In the non-limiting example of the medical environment, an ultrasound system completes this aspect by pushing the study (completed task/event) to a picture archiving and communication system (PACS) archive for generation of results and notifies the program code that the task/event is complete (295). The program code then reserves a current dynamic accession number for a task/event (e.g., study) that has not taken place ([-/.]1), and the program code increments the dynamic accession number for the next study ([-/.]2) (298).

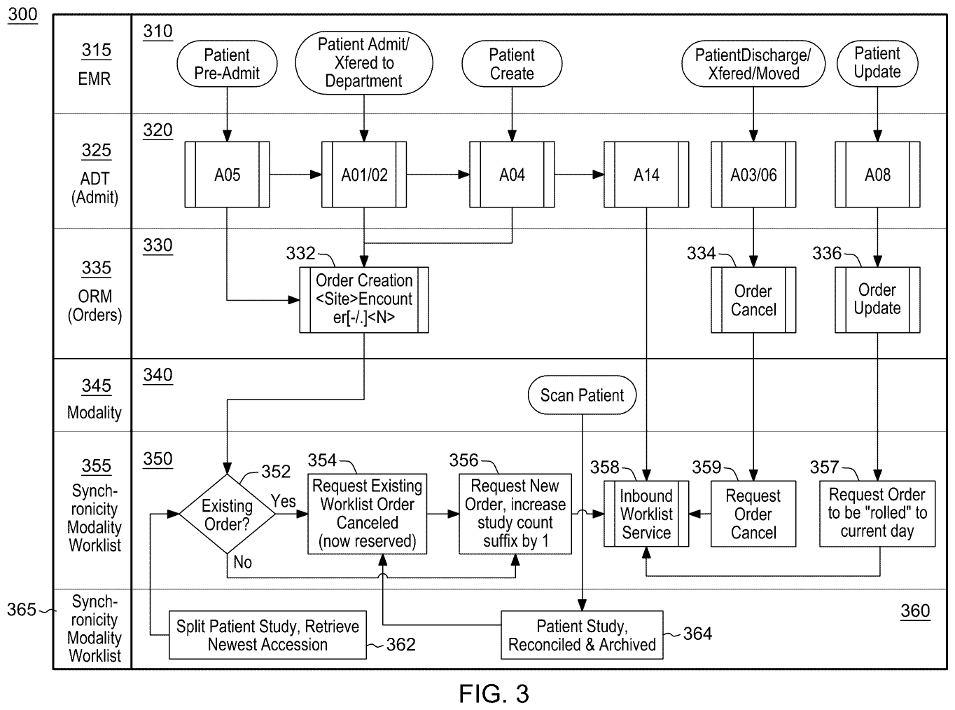

As discussed herein, aspects of embodiments of the present invention can be utilized in a healthcare setting. Thus, to demonstrate certain functionalities of some embodiments of a granular level, FIG. 3, which is a detailed workflow 300, is provided within the context of this setting. This non-limiting example is utilized for illustrative purposes only. FIG. 3 illustrates how specific events that occur in the hospital setting that would trigger the program code in embodiments of the present invention to create the dynamic order numbers with associated dynamic accession numbers and to enforce the integrity of these orders. This example includes Admission/Discharge Transfer System (ADT) system events from electronic medical records (EMRs). FIG. 3 illustrates the interaction of various systems within the computing environment with the program code. These systems can be implemented as any type of module or component in software (e.g., as software instructions that are executable with a processing system), hardware, or combinations thereof, as a standalone application or as a module or component of another device application, and in any type of computing device. The term program code is used throughout this application and can refer to logic executed by both hardware and software, including the implementations noted.

Referring to FIG. 3, various events 310 open patient records in an EMR system 315. The examples of events 310 provided herein are a patient pre-admission, a patient transfer to a different department, the creation of a patient in the system, and the discharge or movement of a patient. The creation of patient records in the EMR system 315, triggers the creation of various messages 320 by the ADT system 325. Based on actions in the EMR system, the program code and other systems within the computing environment are notified that a patient is at the location (see, e.g., FIG. 2, 210). Based on the message 320 from the ADT system 325, orders are entered into an Order system 335. The messages generated by the ADT system 325 are HL7 messages in the depicted example. A05 is a message to pre-admit a patient. A01 is an admission and A05 is a transfer, while A04 is a message to register a patient. As seen in FIG. 3, the A05, A01/02, and A04 messages all prompt the Order system 335 to create an order 332. The A014 message is a pending admission message which is a transmission to the worklist 358. The EMR system 315 action of a patient discharge or a move trigger the ADT system 325 to create a message regarding a discharged visit end message, A03, or a message changing an outpatient to an inpatient, A06. Based on the A08 message to update patient information, the Order system 335 can update the order 336, to move the order to a different day, which the program code populates 359 in the worklist 358. When the HL7 message indicates a discharge (A03) or a change to an inpatient (A06), the Order system 335 can cancel the order 334, and alert the program code of an embodiments of the present invention of this request, which the program code populates 359 in the worklist 358.

The HL7 Order Entry (ORM) message can be the message type utilized to hold information about a request for materials or services. The Order system 335 generates order events 330, including generating at least one order 332 and the program code executing one or more processors generates a dynamic order number and a dynamic accession number. Various actions 350 completed by the program code are related to maintaining the synchronicity of the modality worklist 355 and are thus grouped together for illustrative purposes. The program code obtains the order 332 and determines whether this order already exists 352. If the program code determines that the order already exists, the program code reserves the order 354 and requests/generates a new order 356, which it transmits to a worklist 358. If the program code determines that the order does not exist, the program code requests/generate a new order 356, which it transmits to the worklist 358. To maintain the uniqueness or the numbering utilized within the system, various aspects of the program code can be considered part of a synchronicity application or module 365. Various actions 360 can be completed by the synchronicity application or module 365 to preserve the consistency of the dynamic order and accession numbers, some of which are illustrated in FIG. 3. When the program code splits a given study, the program code generates/obtains a new accession number 362, which it provides to the worklist management 350. The program code then checks whether the order is duplicative 352. When the program code reconciles and archives a completed study 364, the program code reserves 354 the order number utilized for this completed study.

In embodiments of the present invention, when the Order system 335 creates a request to cancel an order 335, the program code obtains this request and transmits the cancellation to the worklist 359. If an order is updated 336, given that the completion will take place beyond the window of time defined for locating duplicative orders, the program code obtains a request for this update, and transmits the request to the worklist 357. Meanwhile, various orders are completed 340 by systems 345 within the computing environment, based on items of the worklist, and upon completion, are transmitted to be reconciled and archived 364.

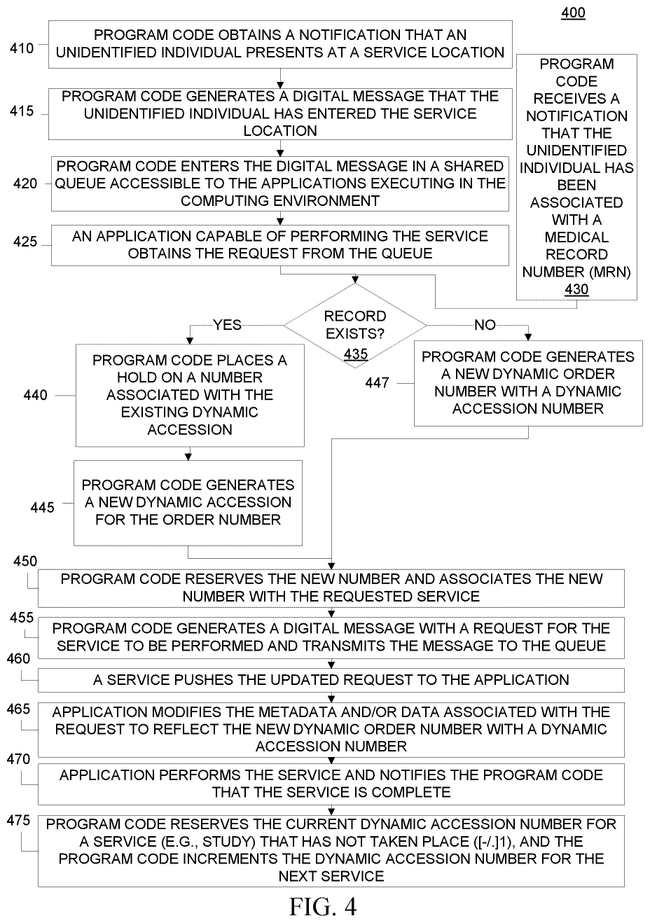

As discussed above, in some embodiments of the present invention, program code executing on one or more processors in embodiments of the present invention can generate a task or event for a shared queue based, preliminarily, on a given consumer entering the service location. However, in some situations, an identity of a consumer is unknown in advance to a service being requested or needed (as determined by the service provider). For example, the consumer may be an unidentified “John Doe” in an emergency room. Given that waiting to provide this service could constitute a risk to the unidentified consumer, in some embodiments of the present invention, a service and its recipient are later reconciled by the program code. FIG. 4 is a workflow 400 that depicts certain aspects of some embodiments of the present invention in this scenario, where the identity of a consumer is known after one or more services have been ordered and/or completed, but not known prior to the one or more services being ordered and/or completed. In this workflow, tasks or services in the shared queue are pushed to modalities in the technical environment to alert the systems or modalities to the identification issues in real-time or close to real-time, to encourage data integrity and reconciliation to occur before the integrity is compromised.

Referring to FIG. 4, in some embodiments of the present invention, the program code obtains a notification that an unidentified individual presents at a service location (410). Program code generates a digital message based on the notification (415). Program code enters the digital message in a shared queue accessible to the applications executing in the computing environment; the digital message is a request for a service to be performed for/on the unidentified individual (420). The request is associated with a temporary identifier. An application capable of performing the service obtains the request from the queue (425).

Asynchronously or synchronously with the application obtaining the service, the program code receives a notification that the unidentified individual has been associated with a medical record number (MRN), providing personally identifiable information about the individual (430). Alerts of an individual being identified to other systems within the computing environment are sent by digital message or through another application, in real-time. The program code determines if a record exists for the now-identified individual during a predefined window of time (e.g., the same business day) (435). Based on determining that a record for the given individual within the defined window of time already exists, the program code places a hold on a number associated with the existing dynamic accession (440). The program code generates a new dynamic accession number and attaches that dynamic accession number to the existing dynamic order number (445). Based on determining there is no duplication, the program code generate a new dynamic order number with a dynamic accession number (447). The program code reserves the new number and associates the new number with the requested service (450). The program code generates a digital message with a request for the service to be performed and transmits the digital message to the queue (455). A service that continuously checks the queue for new messages and updates to messages pushes the updated request to the application that is performing or already performed the service of the update (460). In some embodiments of the present invention, this service is an aspect of the embodiments. In other embodiments of the present invention, the program code initializes this services and/or otherwise communicates with the service, so that the service monitors the queue. Based on obtaining the update, the application modifies the metadata and/or data associated with the request to reflect the new dynamic order number with a dynamic accession number (465). The application completes the service and notifies the program code that the service is complete (470). The program code reserves a current dynamic accession number and the program code increments the dynamic accession number for the next service ([-/.]2) (475). Although in the example, the order and accession numbers associated with a service are modified before the service is performed, in some examples, the number can be modified after performing the service. The reservation of the generated dynamic order and accession numbers avoid duplication. The program code can push the dynamic order number and dynamic accession number to the system(s) associated with performing the task as soon as this information becomes available to preserve the data integrity of the system.

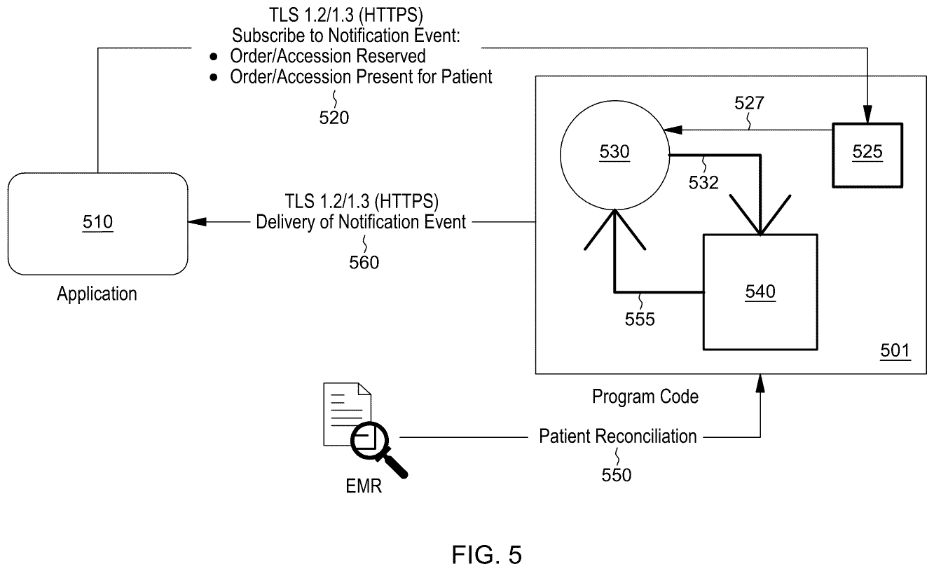

FIG. 5 is a workflow 500 that includes certain details of how program code in embodiments of the present invention can alert modalities to an event (and/or an event update). Certain earlier examples discuss the utilization of digital messaging through the HL7 standard, this example, for illustrative purposes only and not to suggest any limitations, the program code utilizes an alternate method of message delivery, an application procedure interface (API) request. In this example, communications between various elements of the technical elements participating in the workflow 500 communicate via TLS 1.2/1.3 Hypertext Transfer Protocol Secure (HTTPS), a secure web interface. This is a non-limiting examples and provided for illustrative purposes, only. In this example, program code 501 in the depicted embodiment of the present invention performs different functions. For illustrative purposes only, and not to suggest any limitations to the technical architecture of the modules comprising the program code, the program code 501 has been separated into different functions. As will be described below, the program code 501 includes a subscription service 525 that generates a token 530, the generated token 530, and program code that generates the notification to which an application subscribes, via the subscription service 525.

The workflow 500 of FIG. 5 illustrates real-time event subscription and notification. In FIG. 5, an application 510 (e.g., modality) in a computing environment can subscribe to event notifications and based on this subscription, notifications related to these events will be delivered to this subscriber, in real-time. In this example, the application 510 is any application that can request and receive requests conforming to the Hypertext Transfer Protocol (HTTP). As illustrated, the application 510 selects to subscribe to events for when the program code creates dynamic orders and/or a dynamic accessions for patients (520). In this example, the application 510 sends an Application Procedure Interface (API) request (POST) to subscribe to events over Transport Layer Security (TLS) to program code 501 comprising a subscription service 525 in an embodiment of the present invention. A subscription service 525 accepts the subscription and generates a token 530 for the application 510 (527). The token 530 notifies the program code which handles notifications 540 of what subscriptions this application 510 should receive (532).

The technical aspects of the workflow of FIG. 5 are utilized in a scenario where an unknown patient, a “Jane Doe” is admitted to a medical service provider, such as an emergency department. For this reason, this non-limiting example is used in describing aspects of the workflow 500. In this example, a clinician will admit this unknown patient. Once a registrar in the EMR department reconciles the patient's information, providing a true identity for the aforementioned Jane Doe, the program code captures the reconciliation through a digital message (550), and then because of the token 530 indicates that to the application 510 is subscribed to receive this type of reconciliation information (555), the program code sends a notification back to the application (560). In this manner, the program code can notify the clinician, via the application 510, in real-time, that a new order has been placed for the reconciled patient (i.e., Jane Doe). In this example, for each subscribed event an HTTP POST request is sent over TLS to notify the application 510 that one of its subscribed events has triggered.

Embodiments of the present invention include computer-implemented methods, computer program products, and computer systems where program code executed by one or more processors in a computing environment obtains an electronic message indicating an individual is physically present at a service location. The program code generates a new service request for the individual, where the new service request for the individual comprises a unique identifier. The program code transmits the new service request to a shared queue accessible to one or more modalities comprising resources in the computing environment.

In some examples, generating the new service request for the individual includes the program code determining if an existing service request associated with the individual was generated by the processor(s) during a predefined window of time. Based on determining that the existing service request was generated during the predefined window, the program code generates the unique identifier of the new service request based on a unique identifier of the existing service request. The program code generates the unique identifier by: reserving the unique identifier of the existing service request, where a final value of the unique identifier of the existing service request comprises an integer, incrementing the final value of the unique identifier of the existing service request by a predefined value, and associating the incremented unique identifier of the existing service request with the new service request as the unique identifier of the new service request.

In some examples, generating the new service request for the individual includes the program code determining if an existing service request associated with the individual was generated by the processor(s) during a predefined window of time. Based on the program code determining that no existing service request was generated during the predefined window, the program code generates the unique identifier of the new service request, where a final value of the unique identifier of the new service request comprises an integer.

In some examples, the program code obtains a notification from a modality of the one or more modalities that the new service request is complete. The program code reserves the unique identifier of the new service request. The program code increments the final value of the unique identifier of the new service request by the predefined value for use as a unique identifier for a next service request associated with the individual generated during the predefined window of time.

In some examples, the program code identifies at least one modality of the one or more modalities, where the at least one modality comprises technical characteristics enabling the at least one modality to complete the new service request. The program code notifies the at least one modality that the new service request was transmitted to the shared queue. For instance, the program code can notify, via digital message and/or other notifications (including a notification in an application or web interface), a patient health application, an image archiver, and/or a mobile application. In some examples, certain authorized modalities subscribe to receive certain types of notifications. Thus, an authorized subscribed modality would receive this notification.

In some examples, transmitting the new service request to the shared queue and notifying the at least one modality are performed in parallel by the one or more processors.

In some examples, generating the new service request for the individual further comprises: based on determining that the existing service request was generated during the predefined window, the program code collects metadata from the existing service request. The program code incorporates the metadata into the new service request.

In some examples, determining if the existing service request associated with the individual was generated by the one or more processors during the predefined window of time comprises the program code querying one or more databases in the computing environment.

In some examples, the window of time is a calendar day when the individual is physically present at the service location.

In some examples, one or more of the electronic message indicating the individual is physically present at the service location and the transmission of the new service request to the shared queue are in a Health Level Seven (HL7) format.

In some examples, the shared queue comprises a modality worklist.

In some examples, the predefined value is 1.

In some examples, the integer of the new service request comprises an accession number, where the accession number of the new service is a number in an ordered sequence of services requested for the individual during the predefined window.

In some examples, the unique identifier of the new service request comprises a concatenation of values, the values selected from the group consisting of: the service location, a visit number for the individual at the service location, an identifier of the individual, and an identifier of the service.

In some examples, the electronic message indicating the individual is physically present at the service location does not comprise data or metadata identifying the individual, and the method further comprising: the program code associating the new service request with a medical record number in an electronic medical records system in the computing environment; and the program code updating the new service request based on the associating.

In some examples, the program code messages an electronic billing system, to instruct the electronic billing systems to generate a bill for the completed new service request.

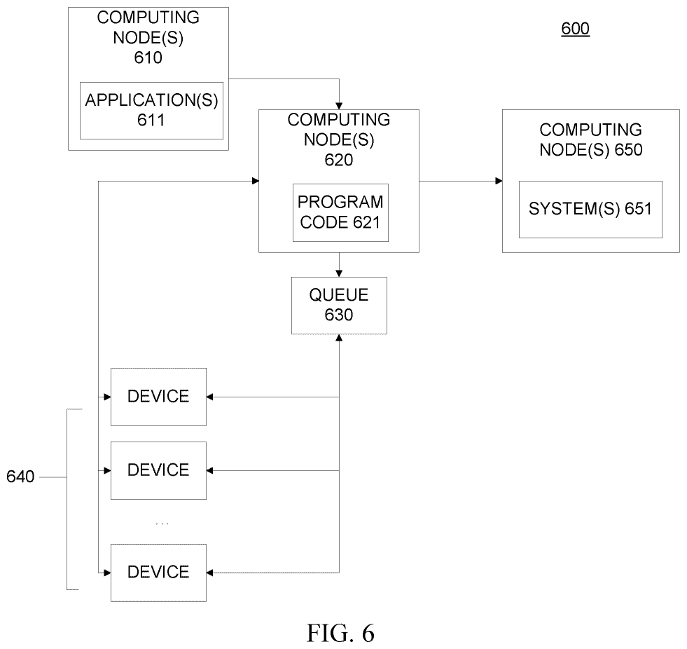

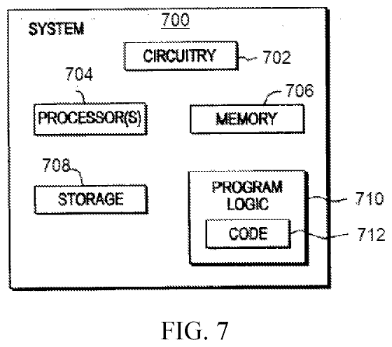

FIG. 6 is a computing environment 600 into which aspects of some embodiments of the present invention can be implemented. Each individual element represented in this technical environment can include one or more computer resources (e.g., FIG. 7, 700). Also, the separation of various nodes herein is provided as an example of a possible configuration. For example, in a technical environment implemented in a medical setting, the same computing resource that executes program code of an embodiment of the present invention, can also execute program code of a system that handles admissions and/or orders. As illustrated herein, one or more computing nodes 610 executing various applications 611, provide one or more computing nodes 620 executing program code implementing aspects of some embodiments of the present invention 621, with data. These data can include a notification that a consumer has entered a premises. The program code sends a message to a queue 630. This communication can occur via a digital message (e.g., HL7), and/or HTTP/HTTPS. The queue 630 is depicted as its own entity herein, but it can be run and maintained by one or more computer resources, and/or it can be maintained on the same computing node(s) 620 whose processors execute the program code 621. This queue 630 is accessible to one or more computing devices 640. These computing devices are or comprise the various modalities which check the queue 630 and/or subscribe to updates to the queue 630. When a given event is complete, a modality of the one or more computing devices 640 notifies the program code 621, and the program code can provide this data to other systems 651 executing on computing nodes 650 in the computing environment 600. For example, the program code 621 can notify a billing system to generate a bill for a given event.

FIG. 7 illustrates a block diagram of a resource 700 in a computer system, such as, which is part of the technical architecture of certain embodiments of the technique. For example, a resource 700 could be connected to or included in the modems utilized in various embodiments of the present invention to send and receive the additional signal over the legacy bus. Additionally or alternatively, certain buses that can be utilized in embodiments of the present invention are themselves computing resources 700. Returning to FIG. 7, the resource 700 can include a circuitry 702 that can in certain embodiments include a microprocessor 704. The computer system 700 can also include a memory 706 (e.g., a volatile memory device), and storage 708. The storage 708 can include a non-volatile memory device (e.g., EEPROM, ROM, PROM, RAM, DRAM, SRAM, flash, firmware, programmable logic, etc.), magnetic disk drive, optical disk drive, tape drive, etc. The storage 708 can include an internal storage device, an attached storage device and/or a network accessible storage device. The system 400 can include a program logic 710 including code 712 that can be loaded into the memory 706 and executed by the microprocessor 704 or circuitry 702.

In certain embodiments, the program logic 710 including code 712 can be stored in the storage 708, or memory 706. Additionally or alternatively, the program logic 710 can be implemented in the circuitry 702. Therefore, while FIG. 7 shows the program logic 710 separately from the other elements, the program logic 710 can be implemented in the memory 706 and/or the circuitry 702. The program logic 710 can include the program code discussed in this disclosure that facilitates the reconfiguration of elements of various computer networks, including those in various figures. Additionally or alternatively, the program logic 710 can include the program code discussed in this disclosure that facilitates preserving data integrity of tasks across a computing environment, as illustrated in FIGS. 2-6.

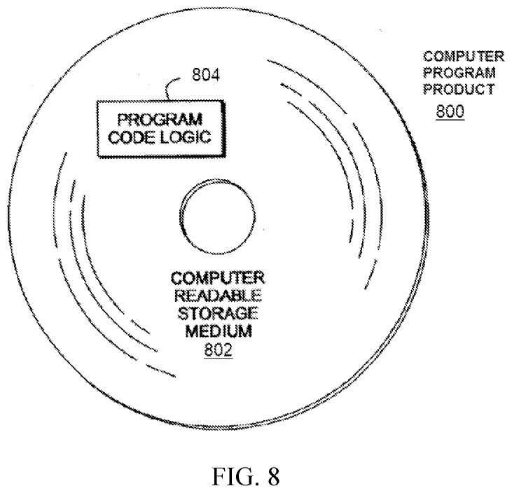

Using the processing resources of a resource 700 to execute software, computer-readable code or instructions, does not limit where this code can be stored. Referring to FIG. 8, in one example, a computer program product 800 includes, for instance, one or more non-transitory computer readable storage media 802 to store computer readable program code means or logic 804 thereon to provide and facilitate one or more aspects of the technique. Examples of non-transitory computer readable storage media 802 include storage memories, such as any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions.

As will be appreciated by one skilled in the art, aspects of the technique can be embodied as a system, method or computer program product. Accordingly, aspects of the technique can take the form of an entirely hardware embodiment, an entirely software embodiment (including firmware, resident software, micro-code, etc.) or an embodiment combining software and hardware aspects that can all generally be referred to herein as a “circuit,” “module” or “system”. Furthermore, aspects of the technique can take the form of a computer program product embodied in one or more computer readable medium(s) having computer readable program code embodied thereon.

Any combination of one or more computer readable medium(s) can be utilized. The computer readable medium can be a computer readable signal medium or a computer readable storage medium. A computer readable signal medium can include a propagated data signal with computer readable program code embodied therein, for example, in baseband or as part of a carrier wave. Such a propagated signal can take any of a variety of forms, including, but not limited to, electro-magnetic, optical or any suitable combination thereof. A computer readable signal medium can be any computer readable medium that is not a computer readable storage medium and that can communicate, propagate, or transport a program for use by or in connection with an instruction execution system, apparatus, or device.

A computer readable storage medium can be, for example, but not limited to, an electronic, magnetic, optical, electromagnetic, infrared or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium can be any tangible medium that can contain or store a program for use by or in connection with an instruction execution system, apparatus, or device.

Any combination of one or more computer readable medium(s) can be utilized. The computer readable medium can be a computer readable signal medium or a computer readable storage medium. A computer readable signal medium can include a propagated data signal with computer readable program code embodied therein, for example, in baseband or as part of a carrier wave. Such a propagated signal can take any of a variety of forms, including, but not limited to, electro-magnetic, optical or any suitable combination thereof. A computer readable signal medium can be any computer readable medium that is not a computer readable storage medium and that can communicate, propagate, or transport a program for use by or in connection with an instruction execution system, apparatus, or device.

Program code embodied on a computer readable medium can be transmitted using an appropriate medium, including but not limited to wireless, wireline, optical fiber cable, RF, etc., or any suitable combination of the foregoing.

Computer program code for carrying out operations for aspects of the technique can be written in any combination of one or more programming languages, including an object oriented programming language, such as Java, Smalltalk, C++ or the like, and conventional procedural programming languages, such as the “C” programming language, PHP, ASP, assembler or similar programming languages, as well as functional programming languages and languages for technical computing (e.g., Python, Matlab). The program code can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). Furthermore, more than one computer can be used for implementing the program code, including, but not limited to, one or more resources in a cloud computing environment.

Aspects of the technique are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions, also referred to as software and/or program code, can also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions can also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various embodiments of the technique. In this regard, each block in the flowchart or block diagrams can represent a module, segment, or portion of code, which comprises one or more executable instructions for implementing the specified logical function(s). It should also be noted that, in some alternative implementations, the functions noted in the block can occur out of the order noted in the figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In addition to the above, one or more aspects of the technique can be provided, offered, deployed, managed, serviced, etc. by a service provider who offers management of customer environments. For instance, the service provider can create, maintain, support, etc. computer code and/or a computer infrastructure that performs one or more aspects of the technique for one or more customers. In return, the service provider can receive payment from the customer under a subscription and/or fee agreement, as examples. Additionally or alternatively, the service provider can receive payment from the sale of advertising content to one or more third parties.

In one aspect of the technique, an application can be deployed for performing one or more aspects of the technique. As one example, the deploying of an application comprises providing computer infrastructure operable to perform one or more aspects of the technique.

As a further aspect of the technique, a computing infrastructure can be deployed comprising integrating computer readable code into a computing system, in which the code in combination with the computing system is capable of performing one or more aspects of the technique.

As yet a further aspect of the technique, a process for integrating computing infrastructure comprising integrating computer readable code into a computer system can be provided. The computer system comprises a computer readable medium, in which the computer medium comprises one or more aspects of the technique. The code in combination with the computer system is capable of performing one or more aspects of the technique.

Further, other types of computing environments can benefit from one or more aspects of the technique. As an example, an environment can include an emulator (e.g., software or other emulation mechanisms), in which a particular architecture (including, for instance, instruction execution, architected functions, such as address translation, and architected registers) or a subset thereof is emulated (e.g., on a native computer system having a processor and memory). In such an environment, one or more emulation functions of the emulator can implement one or more aspects of the technique, even though a computer executing the emulator can have a different architecture than the capabilities being emulated. As one example, in emulation mode, the specific instruction or operation being emulated is decoded, and an appropriate emulation function is built to implement the individual instruction or operation.

In an emulation environment, a host computer includes, for instance, a memory to store instructions and data; an instruction fetch unit to fetch instructions from memory and to optionally, provide local buffering for the fetched instruction; an instruction decode unit to receive the fetched instructions and to determine the type of instructions that have been fetched; and an instruction execution unit to execute the instructions. Execution can include loading data into a register from memory; storing data back to memory from a register; or performing some type of arithmetic or logical operation, as determined by the decode unit. In one example, each unit is implemented in software. For instance, the operations being performed by the units are implemented as one or more subroutines within emulator software.

Further, a data processing system suitable for storing and/or executing program code is usable that includes at least one processor coupled directly or indirectly to memory elements through a system bus. The memory elements include, for instance, local memory employed during actual execution of the program code, bulk storage, and cache memory which provide temporary storage of at least some program code in order to reduce the number of times code must be retrieved from bulk storage during execution.

Input/Output or I/O devices (including, but not limited to, keyboards, displays, pointing devices, DASD, tape, CDs, DVDs, thumb drives and other memory media, etc.) can be coupled to the system either directly or through intervening I/O controllers. Network adapters can also be coupled to the system to enable the data processing system to become coupled to other data processing systems or remote printers or storage devices through intervening private or public networks. Modems, cable modems, and Ethernet cards are just a few of the available types of network adapters.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or steps plus function elements in the descriptions below, if any, are intended to include any structure, material, or act for performing the function in combination with other elements as specifically noted. The description of the technique has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular uses contemplated.

1. A computer-implemented method comprising:
obtaining, by one or more processors in a computing environment, an electronic message indicating an individual is physically present at a service location;
generating, by the one or more processors, a new service request for the individual, wherein the new service request for the individual comprises a unique identifier; and
transmitting, by the one or more processors, the new service request to a shared queue accessible to one or more modalities comprising resources in the computing environment.

2. The computer-implemented method of claim 1, wherein generating the new service request for the individual comprises:
determining, by the one or more processors, if an existing service request associated with the individual was generated by the one or more processors during a predefined window of time;
based on determining that the existing service request was generated during the predefined window, generating, by the one or more processors, the unique identifier of the new service request based on a unique identifier of the existing service request, the generating comprising:
reserving, by the one or more processors, the unique identifier of the existing service request, wherein a final value of the unique identifier of the existing service request comprises an integer;
incrementing, by the one or more processors, the final value of the unique identifier of the existing service request by a predefined value; and
associating, by the one or more processors, the incremented unique identifier of the existing service request with the new service request as the unique identifier of the new service request.

3. The computer-implemented method of claim 1, wherein generating the new service request for the individual comprises:
determining, by the one or more processors, if an existing service request associated with the individual was generated by the one or more processors during a predefined window of time; and
based on determining that no existing service request was generated during the predefined window, generating, by the one or more processors, the unique identifier of the new service request, wherein a final value of the unique identifier of the new service request comprises an integer.

4. The computer-implemented method of claim 1, further comprising;
obtaining, by the one or more processors, a notification from a modality of the one or more modalities that the new service request is complete;
reserving, by the one or more processors, the unique identifier of the new service request; and
incrementing, by the one or more processors, the final value of the unique identifier of the new service request by the predefined value for use as a unique identifier for a next service request associated with the individual generated during the predefined window of time.

5. The computer-implemented method of claim 1, further comprising:
identifying, by the one or more processors, at least one modality of the one or more modalities, wherein the at least one modality comprises technical characteristics enabling the at least one modality to complete the new service request; and
notifying, by the one or more processors, the at least one modality that the new service request was transmitted to the shared queue.

6. The computer-implemented method of claim 4, wherein transmitting the new service request to the shared queue and notifying the at least one modality are performed in parallel by the one or more processors.

7. The computer-implemented method of claim 2, wherein generating the new service request for the individual further comprises:
collecting, by the one or more processors, metadata from the existing service request; and
incorporating, by the one or more processors, the metadata into the new service request.

8. The computer-implemented method of claim 2, wherein determining if the existing service request associated with the individual was generated by the one or more processors during the predefined window of time comprises querying, by the one or more processors, one or more databases in the computing environment.

9. The computer-implemented method of claim 2, wherein the window of time is a calendar day when the individual is physically present at the service location.

10. The computer-implemented method of claim 1, wherein one or more of the electronic message indicating the individual is physically present at the service location and the transmission of the new service request to the shared queue are in a Health Level Seven (HL7) format.

11. The computer-implemented method of claim 1, wherein the shared queue comprises a modality worklist.

12. The computer-implemented method of claim 2, wherein the predefined value is 1.

13. The computer-implemented method of claim 3, wherein the integer of the new service request comprises an accession number, wherein the accession number of the new service is a number in an ordered sequence of services requested for the individual during the predefined window.

14. The computer-implemented method of claim 1, wherein the unique identifier of the new service request comprises a concatenation of values, the values selected from the group consisting of: the service location, a visit number for the individual at the service location, an identifier of the individual, and an identifier of the service.

15. The computer-implemented method of claim 1, wherein the electronic message indicating the individual is physically present at the service location does not comprise data or metadata identifying the individual, the method further comprising:
associating, by the one or more processors, the new service request with a medical record number in an electronic medical records system in the computing environment; and
updating, by the one or more processors, the new service request based on the associating.

16. The computer-implemented method of claim 4, further comprising:
messaging, by the one or more processors, an electronic billing system, to instruct the electronic billing systems to generate a bill for the completed new service request.

17. A computer program product comprising:
a computer readable storage medium readable by one or more processors of a shared computing environment comprising a computing system and storing instructions for execution by the one or more processors for performing a method comprising:
obtaining, by the one or more processors in a computing environment, an electronic message indicating an individual is physically present at a service location;
generating, by the one or more processors, a new service request for the individual, wherein the new service request for the individual comprises a unique identifier; and
transmitting, by the one or more processors, the new service request to a shared queue accessible to one or more modalities comprising resources in the computing environment.

18. The computer program product of claim 17, wherein generating the new service request for the individual comprises:
determining, by the one or more processors, if an existing service request associated with the individual was generated by the one or more processors during a predefined window of time;
based on determining that the existing service request was generated during the predefined window, generating, by the one or more processors, the unique identifier of the new service request based on a unique identifier of the existing service request, the generating comprising:
reserving, by the one or more processors, the unique identifier of the existing service request, wherein a final value of the unique identifier of the existing service request comprises an integer;
incrementing, by the one or more processors, the final value of the unique identifier of the existing service request by a predefined value; and
associating, by the one or more processors, the incremented unique identifier of the existing service request with the new service request as the unique identifier of the new service request.

19. The computer program product of claim 17, wherein generating the new service request for the individual comprises:
determining, by the one or more processors, if an existing service request associated with the individual was generated by the one or more processors during a predefined window of time; and
based on determining that no existing service request was generated during the predefined window, generating, by the one or more processors, the unique identifier of the new service request, wherein a final value of the unique identifier of the new service request comprises an integer.

20. A computer system comprising:
a memory;
one or more processors in communication with the memory;
program instructions executable by the one or more processors in a shared computing environment of a computing system via the memory to perform a method, the method comprising:
obtaining, by the one or more processors in a computing environment, an electronic message indicating an individual is physically present at a service location;
generating, by the one or more processors, a new service request for the individual, wherein the new service request for the individual comprises a unique identifier; and transmitting, by the one or more processors, the new service request to a shared queue accessible to one or more modalities comprising resources in the computing environment.
